(19) United States
(12) Patent Application Publication
(10) Pub. No.: US 2008/0026066 A1
(43) Pub. Date: Jan. 31, 2008

(54) Liquids Containing Suspended Glass Particles

(75) Inventor: Bruce Joseph ROSER, Cambridge Cambridgeshire (GB)

Correspondence Address: BROOKS KUSHMAN P.C./FGTL, 1000 TOWN CENTER, 22ND FLOOR, SOUTHFIELD, MI, 48075-1238, US

(73) Assignee: CAMBRIDGE BIOSTABILITY LIMITED, Cambridge Cambridgeshire (GB)

(21) Appl. No.: 10/599,928

(22) PCT Filed: Apr. 13, 2005
(86) PCT No.: PCT/GB05/50050

§ 371 Date: Oct. 13, 2006

(30) Foreign Application Priority Data
Apr. 13, 2004 (GB) 0408199.8
Mar. 7, 2005 (GB) 0504501.8

(51) Int. Cl. A61K 39/00; A61K 9/14

(52) U.S. Cl. 424/489; 424/184.1

(57) ABSTRACT

Present proposals to use perfluorocarbons as a medium to suspend glass particles presents the problem of aggregation of the particles within the suspending medium. Overcoming this problem requires careful particle sizing and density matching techniques. An additional disadvantage of the large scale use of perfluorocarbons is their contribution to global warming. The inventor has realised that by replacing perfluorocarbons with the more environmentally friendly fluorinated ethers such as hydrofluoroethers or hydrofluoropolyethers a long lasting suspension of glass particles can be achieved without the need for such rigorous particle sizing or density matching processes.

This invention relates to a formulation comprising an active ingredient preserved in particles of a glassy or amorphous substance suspended in a liquid.

It is well known that sugar glass has an ability to preserve certain organic, biological, botanical and protein materials and there is a considerable amount of literature devoted to theoretical proposals for using this property of sugar glass to preserve pharmaceutical products, particularly vaccines. Other glassy substances have been shown to have a similar preservative effect.

Because the most commonly accepted method of administering vaccines is by injection it has been proposed, eg in patent specification WO 02/32402 (Roser) to suspend particles of water soluble glass, containing the vaccine, in a liquid (a perfluorocarbon such as perfluorodecalin) so as to create an injectable formulation. Perfluorocarbons were proposed because they are very stable and know as being safe for pharmaceutical and medical uses. It was also proposed in patent specification PCT WO 02/32402 to increase the density of the glass by adding calcium phosphate (density about 2.7 to 2.8) to the sugar glass (density about 1.5) so as to produce particles matched to the 1.97 density value of the liquid in which they were to be suspended; thereby keeping them in suspension.

The above techniques, show great promise, but the complete stability of perfluorocarbons mean that they are persistent in the troposphere and, if used in large amounts could actually contribute to global warming. In addition hydrophilic glass microsphere particles show a slight tendency to aggregate in perfluorocarbons, which are intensely hydrophobic.

According to this invention there is provided a formulation comprising an active ingredient preserved in glassy or amorphous particles, the particles being suspended in a liquid in which at least one component comprises a hydrofluoroether, perfluroether, hydrofluoroamine, perfluoroamine, hydrofluorothioether, perfluorothioether hydrofluoropolyether, perfluorpolyether or a general formula

R1-X—R2 or

R1-X-(CF2Y)n(CF2CF2Z)m-R2 or

R1-[(X—CF—R2)n-(X—CF2)m]OR3

where X, Y and Z are defined as O (oxygen), an ether, NR3 (N=nitrogen), an amine or S (sulphur); and each of R1, R2 and R3 are defined as a non-fluorinated, partially fluorinated or fully fluorinated alkyl, cycloalkyl, aryl or arylalkyl group or an organic functional group, halogen group or cyano group.

Preferably, hydrofluoroethers or hydrofluoropolyethers are considered ideal and accordingly there is provided a formulation comprising an active ingredient preserved in glassy or amorphous particles, the particles being suspended in a liquid comprising a hydrofluoroether or hydrofluoropolyether.

The inventors discovered that when mixed glass particles were added to a hydrofluoroether or hydrofluoropolyether, they dispersed astonishingly easily to form a milky suspension with little or no signs of clumping of the glass particles even after the suspension had been left for some time.

The inventors have now developed the theory that the glass particles have a hydrophilic surface whilst the perfluorocarbons, previously used, are intensely hydrophobic. For this reason, in the earlier experiments with perfluorocarbons, it is now believed that the glass particles had a tendency to clump together because they are repelled by the hydrophobic nature of the perfluorocarbon. Fluorinated ethers, behave somewhat more like a detergent, facilitating dispersion of the particles.

A number of fluorinated ethers are presently being administered as anaesthetic agents via inhalation during surgical procedures. The relatively large quantities (up to 200 gms) which are used during surgical procedures indicates the low-toxicity of the group.

Additionally, their densities are ideally matched to the densities of glasses used in the formulations described above. For example, referring to the designations of 3M Limited:
HFE 7500 has a density of 1.61,
HFE 7200 has a density of 1.43, and
HFE 7100 has a density of 1.52.

These values are, co-incidentally similar to the density of sugar glass, which is about 1.5.

An additional benefit of using the invention is that fluorinated ethers, whilst being highly stable in normal conditions, are unstable when exposed to strong ultraviolet radiation such as is present in the stratosphere. This avoids a problem associated with perfluorocarbons which are known to contribute to the damaging “greenhouse” effect when released into the atmosphere after use.

Yet another advantage of the invention is that fluorinated ethers are relatively inexpensive and are readily available at a high degree of purity, greater than 98%. This compares with PFCs for which a typical example might have a purity of only about 55%.

Because fluorinated ethers are so well matched with the glasses, it has become possible to adopt a new approach to density matching. Previously, the glass was formulated, by use of additives, to match its density to that of the liquid PFC. However, it now becomes unnecessary to constrain the selection of the glass according to the need to achieve the correct density. The invention makes it possible to select the ideal glass/active ingredient composition; and then to mix a fluorinated ether possibly with the addition of small quantities of PFCs or other liquids so as to match the density of the liquid to the density of the particles. It even becomes practicable to take ready-made compositions of active ingredient preserved in a glassy substance; to grind it into particles and then to suspend it in a liquid matched to the density of the particles.

The densities of the particles and of the liquid do not have to be identical. However, they should be sufficiently close that Brownian movement or other thermodynamic influences keep the particles in suspension.

Because the particles have been found to disperse so effectively in fluorinated ethers and other liquids referred to above, the need to make the particles as small as possible, so as to maintain a suspension, is now not as acute as before. Specialist, modified spray drying techniques, which were previously thought by the inventors to be needed in order to achieve small particle size, are now unnecessary although the standard commercial spray drying process is still one possible technique for making the particles. However, alternative methods such as freeze drying or grinding would now also be practicable. It is only necessary that the particles should be sufficiently small to permit passage through a hypodermic syringe.

It is envisaged that the invention will normally be employed for the formulation of vaccines, therapeutic proteins or other medications for injection through the skin of a patient. However, other uses for the invention may be possible, eg for medicinal liquids which are administered orally or inhaled after atomising. It is also possible that there may be non-medicinal uses for the invention which is generally applicable to any situation where it is desired to preserve a biologically active material in a glassy solid and where there is a need for the composition to be presented in liquid form.

One way of performing the invention will now be described.

Sterile, bulk liquid hepatitis B vaccine with aluminium hydroxide adjuvant was obtained from Panacea Biotech of Delhi. This was mixed with sterile colloidal calcium phosphate suspension and raffinose solution in the correct proportions to give a single adult dose of 10 μg vaccine in 50 milligrams of total solids. The proportion of calcium phosphate to raffinose was calculated to give solid glass particles with a density matching that of the hydrofluoroether HFE 7,500 of 1.61 Kg/L. While being constantly stirred by a magnetic stirrer, this suspension was pumped through a two fluid nozzle at the rate of 2 ml per minute with a nozzle gas flow of 2.5 Kg/hr. The resulting droplets were dried in the chamber of a GEA Niro SD Micro spray with a heated air flow of 30 Kg per hour. The outlet temperature was maintained at 90° C. by regulating the inlet temperature keeping the feed flow rate constant. Product was collected in a sterile bottle and transferred to a laminar flow hood with class 100 air flow. Sterile HFE 7,500 was added at the rate of 1 ml per 100 mg of powder and agitated in a frequency sweep ultrasonic bath for 10 min to fully disperse the microspheres. In the flow hood, the liquid was dispensed in 0.6 ml volumes into sterile 2 ml serum vials, plugged with neoprene stoppers and sealed with aluminium caps. The vaccine vials were used to set up a study of the in vitro stability of the vaccine at various storage temperatures.

1. A formulation comprising an active ingredient preserved in glassy or amorphous particles, the particles being suspended in a liquid in which at least one component comprises a hydrofluoroether, perfluroether, hydrofluroamine, perfluoroamine, hydrofluorothioether, perfluorothioether hydrofluoropolyether, perfluorpolyether or a general formula
R1-X—R2 or
R1-X-(CF2Y)n(CF2CF2Z)m-R2 or
R1-[(X—CF—R2)n-(X—CF2)m]OR3
where X, Y and Z are defined as O (oxygen), and ether, NR3 (N=nitrogen), an amine or S (sulphur); and each of R1, R2 and R3 are defined as a non-fluorinated, partially fluorinated or fully fluorinated alkyl, cycloalkyl, aryl or arylalkyl group or an organic functional group, halogen group or cyano group.

2. A formulation according to claim 1 in which the particles contain a sugar glass or a glass which is a mixture of sugar, metal carboxylate, amino acid or calcium phosphate or any combination of these.

3. A formulation according to claim 1 in which the particles have a density which is matched to the density of the liquid sufficiently closely that the particles will remain in suspension under normal conditions.

4. A formulation according to claim 1 in which the liquid contains different components specified in claim 1 mixed in proportions to give a required density.

5. A formulation according to claim 1 in which the liquid contains a perfluorocarbon mixed with one or more components specified in claim 1.

6. A formulation according to claim 1 in which the active ingredient is a vaccine.

7. A formulation according to claim 1 in which the particles are made by spray drying.

8. A formulation according to claim 1 in which the particles are made by freeze drying.

9. A formulation according to claim 1 in which the particles are made by grinding.

10. A method of making a formulation according to claim 4 including the step of selecting liquids to give the required density matching properties and mixing them with the particles.

11. A formulation comprising an active ingredient preserved in glassy or amorphous particles, the particles being suspended in a liquid comprising a hydrofluoroether.
